(19) United States
(12) Patent Application Publication
(10) Pub. No.: US 2026/0284384 A1
(43) Pub. Date: Sep. 24, 2026

(54) A DELIVERY SYSTEM FOR PLACEMENT OF A LEADLESS PACEMAKER DEVICE IN A HUMAN BODY

(71) Applicant: BIOTRONIK SE & Co. KG, Berlin (DE)

(72) Inventors: Brian M. TAFF, Portland, OR (US); Madeline Anne MIDGETT, Portland, OR (US); R. Hollis WHITTINGTON, Portland, OR (US); Hannes KRAETSCHMER, West Linn, OR (US)

(73) Assignee: BIOTRONIK SE & Co. KG, Berlin (DE)

(21) Appl. No.: 19/475,838

(22) PCT Filed: Apr. 4, 2024
(86) PCT No.: PCT/EP2024/059154

§ 371 Date: Oct. 16, 2025

Related U.S. Application Data
(60) Provisional application No. 63/460,715, filed on Apr. 20, 2023.

(30) Foreign Application Priority Data
May 23, 2023 (EP) 23174710.6

(51) Int. Cl. A61N 1/05; A61M 25/01; A61N 1/362; A61N 1/375

(52) U.S. Cl. CPC A61N 1/0573; A61N 1/362; A61N 1/3756; A61M 2025/0166; A61N 2001/058; A61N 1/37512

(57) ABSTRACT

Delivery system for placement of a leadless pacemaker device in a body including: a catheter device for insertion into the body having a lumen and a distal end for insertion into the body, the lumen configured to receive a leadless pacemaker device in the distal end; first and second mapping electrodes disposed in the distal end for sensing a mapping signal between the first and second mapping electrodes; the first mapping electrode arranged on a mapping extension protruding from a sidewall of the distal end towards a central axis extending in a longitudinal direction of the catheter device; the mapping extension movable with respect to the sidewall between non-mapping and mapping positions, wherein an angle between the mapping extension and a virtual plane extending perpendicular to the central axis is bigger than 5° in the non-mapping position and in a range of from 0° to 5° in the mapping position.

### CROSS-REFERENCE TO RELATED APPLICATIONS

This application is the United States National Phase under 35 U.S.C. § 371 of PCT International Patent Application No. PCT/EP2024/059154, filed on Apr. 4, 2024, which claims the benefit of European Patent Application No. 23174710.6, filed on May 23, 2023, and U.S. Provisional Patent Application No. 63/460,715, filed on Apr. 20, 2023, the disclosures of which are hereby incorporated by reference herein in their entireties.

### TECHNICAL FIELD

The present invention relates to a delivery system for placement of a leadless pacemaker device in a human body according to the preamble of claim 1, to a pacemaker arrangement comprising such a delivery system according to the preamble of claim 12, as well as to a method of mapping a tissue region for identifying an appropriate implantation site for a leadless pacemaker device according to the preamble of claim 15.

### BACKGROUND

In recent years, leadless pacemakers have received increasing attention. Leadless pacemakers, in contrast to pacemakers implanted subcutaneously using leads extending transvenously into the heart, avoid leads in that the pacemaker device itself is implanted into the heart. The pacemaker typically has the shape of a capsule and is intended for implantation into cardiac tissue, in particular into the wall of the right ventricle. Such leadless pacemakers exhibit the inherent advantage of not using leads, which can eliminate risks for the patient involved with leads transvenously accessing the heart, such as the risk of pneumothorax, lead dislodgement, lead endocarditis, venous thrombosis and the like.

Leadless pacer implantation procedures are slowly becoming more routine as leadless systems have begun to erode portions of the traditional pocket-based implantable pace generator (IPG) market and more doctors and centers are becoming familiar with leadless pacing technologies. As leadless systems are larger in mass and volume than lead tips but still demand safe/robust anchoring within a patient's heart, the footprint of their mechanical anchors is substantially larger than that of traditional pocket-based leaded IPG systems. This footprint tends to introduce more insertion points in the patient anatomy with a greater overall capacity to introduce scarring and/or pericardial effusion subject to problematic perforations. There is therefore a need to minimize the amount of physical insult introduced to the patient's anatomy as part of the process of locating a reliable anchoring site where the implant's tip electrode can effectively access viable electrical engagements with the patient's cardiac conduction system.

No solutions presently exist in commercially available leadless pacemaker systems for improving the baseline implantation procedures need to minimize the number of repositioning instances exercised (or avoiding them altogether) as part of leadless device installation. At best, physician experience with the technology may assist in the process but this familiarity and mastery is still not a true remediation for the undesirable aspects associated with implantation procedures.

US 2020/0338356 A1 describes a mapping catheter system for use with a leadless pacemaker focused on His bundle stimulation.

U.S. Pat. No. 10,874,850 B2 describes another device for delivering an implantable medical device, wherein the delivery device comprises a first electrode inside a delivery bay and a second electrode outside the delivery bay for measuring an impedance between the inside and the outside of the delivery bay.

The present disclosure is directed toward overcoming one or more of the above-mentioned problems, though not necessarily limited to embodiments that do.

### SUMMARY

It is an object of the present invention to provide a delivery system that enables the identification of an appropriate implantation site for a leadless pacemaker device without mandating that a clinician first scars or perforates untoward amounts of cardiac or other tissue.

This object is achieved with a delivery system for placement of a leadless pacemaker device in a human body having the claim elements of claim 1. Such a delivery system comprises a delivery catheter 4 insertion into a human body. The catheter device has a lumen and a distal end region to be inserted into the human body. In this context, the lumen is configured to receive a leadless pacemaker device in the distal end region of the catheter device.

The delivery system further comprises a first mapping electrode and a second mapping electrode disposed at the distal end region of the catheter device. In a mapping mode, the first and second mapping electrodes serve for sensing a mapping signal between the first mapping electrode and the second mapping electrode.

The first mapping electrode is arranged on a mapping extension. This mapping extension protrudes from a sidewall of the distal end region of the catheter device towards a central axis of the catheter device. The central axis extends in a longitudinal extension direction of the catheter device. Typically, the sidewall extends parallel to the longitudinal axis.

The mapping extension is movable with respect to the sidewall of the distal end region of the catheter device between a non-mapping position and a mapping position. An angle between the mapping extension and a virtual plane extending perpendicular to the central axis is bigger than 5° in the non-mapping position. The virtual plane intersects the sidewall of the delivery catheter at that site at which the mapping extension is connected to the sidewall. In an embodiment, the angle lies in a range of from 5° to 90°, in particular of from 10° to 85°, in particular of from 15° to 80°, in particular of from 20° to 70°, in particular of from 25° to 60°, in particular of from 30° to 50°, in particular of from 35° to 40°. In the mapping position, the angle between the mapping extension and the virtual plane lies in a range of from 0° to 5°, in particular of from 1° to 4°, in particular of from 2° to 3°. I.e., in the mapping position, the mapping extension is arranged essentially perpendicular to the sidewall of the distal end region of the catheter device, if the sidewall extends essentially parallel to the longitudinal axis (and thus perpendicular to the virtual plane).

The second electrode is arranged on an outside of the sidewall in the distal end region of the catheter device.

In contrast to prior art solutions, it is not necessary that the anchors of the leadless pacemaker device be deployed for mapping the tissue to find an appropriate implantation site. Rather, the leadless pacemaker received within the lumen of the distal end region of the catheter device can be kept in its delivery position when performing a mapping of the tissue to find an appropriate implantation site.

Furthermore, in contrast to other prior art solutions, the mapping signal is much better correlated to later sensing and stimulation signals detected or, respectively, applied by the leadless pacemaker device to be implanted by the delivery system. This is because the first mapping electrode will contact the tissue at a site at which the implant's sensing and pacing tip electrode will ultimately contact the patient's tissue, whereas prior art either demands that the implant be extended from the catheter or acquires the mapping data at a location that is guaranteed to be physically offset from where the implant's sensing and pacing tip electrode will ultimately contact the patient's tissue. In addition, only the first mapping electrode is placed on the mapping extension, wherein the second mapping electrode is placed on the outside of the distal end region of the catheter device. Thus, the first and second mapping electrodes are spaced like the tip and return electrode of an implant to be delivered by the delivery system. Thus, the physical distance between the two mapping electrodes matches the separation of the tip and return electrode on the implant to be delivered. Consequently, the catheter device realizes the same sensing vector and therefore is able to collect signaling that will better match what would be observable once the implant is installed into the myocardium. Prior art solutions using two mapping electrodes on a mapping extension do not appropriately consider the sensing vector applied by the leadless pacemaker device to be implanted. Rather, some of the prior art solutions make use of two ring electrodes on a delivery catheter for mapping purposes that do, however, not properly reflect the effective impedances and current densities realized between the electrodes of the leadless pacemaker to be implanted by such catheter device. In contrast, when the relative sizing of the first mapping electrode and the second mapping electrode is aligned with the sizing of the tip and ring electrode found on the implant to be delivered by the delivery system, the effective impedances between the mapping electrodes as well as the realizable current densities at the first mapping electrode/tissue interface better mimic the implant to both collect signaling matched to what the implant would sense and also better mimic the localization of stimulated output as would be administered by the implant.

In contrast to prior art solutions, the specific arrangement of the first mapping electrode and the second mapping electrode allows to not only record an intracardiac electrogram (IEGM), but rather measure the impedance between the two mapping electrodes and/or to perform a pacing capture threshold test with the mapping electrodes of the delivery system prior to implanting a leadless pacemaker. Sensing electric cardiac signals (such as P waves, R waves or an IEGM), measuring an impedance between the first mapping electrode and the second mapping electrode, and determining how effective a pacing output is (pacing capture threshold test) are typical mapping tasks performed in the course of mapping tissue to identify an appropriate implantation site. Since the size and separation of the catheter-based mapping electrodes (i.e., the first and second mapping electrodes) matches those found on the implant to be delivered, the delivery system ensures access to data commensurate with what the implant could collect but prior to enforcing mechanical engagements between the implant's fixation mechanism and patient heart tissue.

The present invention is particular advantageous for the implantation of a leadless pacemaker suitable for conduction system pacing. Conduction system pacing (CSP) is a therapy that involves the placement of a leadless pacemaker along different sites of the cardiac conduction system with the intent of overcoming sites of atrioventricular (AV) conduction disease and delay, thereby providing a pacing solution that results in more synchronised biventricular activation. Leadless pacemaker placement for CSP can be targeted either at the bundle of His, known as His bundle pacing (HBP), or at the region of the left bundle branch (LBB), known as LBB pacing (LBBP), or at the region of the right bundle branch (RBB). CSP could be used to achieve cardiac resynchronisation in patients with heart failure with reduced ejection fraction and inter-ventricular dyssynchrony. The use of the delivery system or method according to the present invention eases the placement of the leadless pacemaker at the His bundle, LBB and/or RBB. The mapping signal derived by the delivery catheter allows a more reliable identification of the conduction system in the region of the implantation site. The leadless pacemaker could be placed exactly at the intended part in the conduction system.

The delivery system enables a reduction of the number of holes poked into the patient's tissue (such as the patient's myocardium) as part of leadless pacemaker implantation procedures. Optimally, the delivery system fully avoids poking such holes into the patient's tissue. Furthermore, the delivery system opens up the possibility for reducing implantation procedure durations as fewer retry steps are needed to position the leadless pacemaker at an appropriate implantation site.

Since the mapping extension is, in its non-mapping position, angularly arranged with respect to the virtual plane (which arrangement is typically achieved by a spring load forcing the mapping extension into its non-mapping position) and needs to be pressed against tissue to be transferred into its mapping position, in which the mapping extension is essentially perpendicular to the central axis and typically perpendicular to the sidewall of the distal end region of the catheter device, the mapping extension firmly abuts the tissue to be mapped against which the catheter device is pressed.

Typically, the mapping extension is biased into the non-mapping position. Thus, as long as there is no tight contact between the catheter device and the tissue to be mapped, the mapping extension will adopt its non-mapping position. Upon pushing the catheter device against the tissue to be mapped, the mapping extension is transferred to its mapping position in which it exerts a slight pressure against the tissue due to this pretensioning. This pretensioning additionally applies a tight yet atraumatic contact between the mapping extension (and thus the first mapping electrode) and the tissue to be mapped. Thus, the pretensioned mapping extension is a reliable means for ensuring that the first mapping electrode is, in the mapping position of the mapping extension, in close physical contact with the patient's heart wall, if the tissue to be mapped forms part of the patient's heart wall.

With a distal terminus of the catheter device (i.e., the catheter tip) pressed against the tissue, the catheter device itself can be used, in an embodiment, to acquire insights on the quality of the implantation location by serving as a vehicle for executing sensing, impedance, and pacing capture threshold testing routines without requiring that the implant be anchored in tissue. The catheter device itself may support the execution of such implantation site evaluations either on its own as a standalone capability where embedded displays (e.g., LCDs) report the implantation site conditions or there may be a link (e.g., by means of a cable) to a programmer device where the follow-up test procedures are executed by the catheter subject to GUI-based instruction from the clinician using the programmer device. The latter embodiment can readily be installed as part of the core programmer device application used during follow-up and even be facilitated by guided workflow procedures. In the end, the user is given a possibility to determine whether or not a site within the heart is promising for implantation of a leadless pacemaker and a possibility for reducing the number of injury sites within the heart prior to ultimately installing a leadless pacemaker. In an embodiment, the delivery system is configured to sense a mapping signal between the first mapping electrode and the second mapping electrode. This mapping signal is, in an embodiment, at least one of a sensing signal, an impedance signal, and a pacing capture threshold signal. As already outlined above, these mapping signals can be obtained with the delivery system without deploying a leadless pacemaker device being received within the lumen of the distal end region of the catheter device of the delivery system.

To give an example, the delivery system can be used for the execution of the same follow-up tests (sensing, impedance, and pacing capture threshold) as are standard in implantable pace generator devices (leadless or pocket-based), but performing such execution prior to interfacing the implant's mechanical anchors with the patient's cardiac tissue (i.e., while the implant still resides within the “protector cup” of the delivery system in the distal end region of the implantation catheter). The critical aspect here is that the tests occur while the implant anchors reside within the implantation catheter. In an embodiment, the follow-up test execution is run by the catheter itself (or by a programmer interfacing with the catheter). In another embodiment, the other unique elements introduced as a part of the catheter design cooperate with the implant to support having the implant execute the pre-implantation follow-up tests.

To give background, the follow-up tests common to implantable pace generator devices both historical and present (and therefore also the follow-up tests for which the performance of the present delivery system is configured) electrically assess the implant's ability to engage with the patient's cardiac conduction system and myocardial depolarization potential. They may be run in any order as the execution of one does not impact the results of another and some clinicians may even elect to skip one of the primary three comprising the suite. In no particular order, the follow-up tests comprise at least one of 1.) a sensing test, 2.) an impedance test, and 3.) a pacing capture threshold test.

The sensing test executes by momentarily reducing the pacing rate of therapy administered to the patient in hopes that the patient's intrinsic rhythm can dominate. During this intrinsic output from the heart itself, the P-wave (atrial contractions) and R-wave (ventricular contractions) amplitudes can be assessed and quantified to give the clinician guidance on the implant's capacity to measure critical inputs needed for marker generation that ensures proper pacemaker timer and rate management operation. Interestingly, in an implantation scenario, the implant itself is nominally in a non-pacing state and therefore, within such use cases, reducing the pacing rate of therapy is not normally necessary for preforming the sensing test as no therapy is being administered that might otherwise need throttling.

The impedance test assesses how much loading the electrical pathway between the implant's stimulation cathode and return anode presents to the pacing output of the device. This loading if too low can shorten product longevity in problematic ways.

The pacing capture threshold test determines what pacing voltage and pulse widths are sufficient to ensure that stimulation from the implant will be successful in producing contractions of the chamber in which the pacer resides. Especially in leadless pacers, this output needs to be reduced to the lowest amplitude, but safe settings possible to avoid wasting energy and shortening the product's longevity.

In an embodiment, the mapping extension is made from the same material as the sidewall of the distal end region of the catheter device. An appropriate material is a biocompatible polymer. In an embodiment, the mapping extension is made from a shape memory material, such as Nitinol. Then, it is particularly easy to manufacture the mapping extension with a pretensioning in a desired direction.

In an embodiment, the mapping extension comprises i) an insulating element that surrounds all but the very tip of the mapping extension and ii) a conductive electrical connection that runs from the exposed tip back to the catheter device and/or the tip electrode of an implant received within the catheter device. Thus, the mapping extension may be designed akin to a wire that has insulation on most of its length excepting an exposed distal tip.

In an embodiment, the first mapping electrode is the only electrode being present on the mapping extension. A second electrode on the mapping extension is not necessary since the second mapping electrode serving as return electrode (counter electrode) is located on the outside of the sidewall in the distal end region of the catheter device. I.e., the second mapping electrode is positioned proximally from a distal terminus of the catheter device. The position of the second mapping electrode matches the position of a leadless pacemaker device located inside the lumen of the catheter device in a state to be delivered to the intended site of implantation.

In an embodiment, the first mapping electrode is sized to match the size of a tip electrode of the leadless cardiac pacemaker to be implanted by the delivery system.

In an embodiment, the second mapping electrode (serving as return electrode for the first mapping electrode) is designed as a ring electrode that is sized to match the size of the return electrode of the implantable leadless pacemaker to be implanted by the delivery system. Then, an electric signal sensed or applied between the first mapping electrode and the second mapping electrode will very closely match a corresponding signal between the tip electrode and the return electrode of the implantable leadless pacemaker to be implanted by the delivery system.

In an embodiment, the catheter device is configured to perform the described mapping tests independently on other elements of the delivery system or other additional devices, i.e., in form of a stand-alone catheter behavior.

In another embodiment, the catheter device serves as conduit and administrator for the execution of such mapping test instructed by a separate programming device being operatively coupled to the catheter device. Such a programming device can form part of the delivery system or can be realized separate from the delivery system.

In an embodiment, the first mapping electrode and the central axis of the catheter device intersect at least in the mapping position of the mapping extension. Thus, at least in the mapping position, the first mapping electrode is located in the region of the central axis, i.e., in the same region in which a tip electrode of the leadless pacemaker to be implanted by the delivery system is located when the implantable leadless pacemaker is received within the lumen of the distal end region of the catheter device. Expressed in other words, the location of the first mapping electrode in its mapping position closely matches (or is even identical to) the location of the tip electrode of the implantable leadless pacemaker to be implanted by the delivery system after implantation. Consequently, the values of mapping signals (resulting from sensing events, impedance measurements and/or pacing capture threshold measurements) measured between the first mapping electrode and the second mapping electrode closely match (or are optimally identical to) the values of signals resulting from sensing events, impedance measurements and/or pacing capture threshold measurements performed by the implantable leadless pacemaker after implantation. Expressed in yet other words, the physiologic conditions under which the mapping signals and signals after implantation are obtained are closely similar or even identical to each other.

In an embodiment, the mapping extension protrudes from a distal terminus of the catheter device inclined to the longitudinal extension direction of the catheter device in the non-mapping position. At the same time, the mapping extension is flush with the distal terminus of the catheter device in the mapping position. Thus, the mapping extension is movable in an area distal of the distal terminus of the catheter device (i.e., distally in front of the distal terminus of the catheter device).

In an embodiment, a distance between the first mapping electrode and the second mapping electrode in the mapping position of the mapping extension equals a distance between a tip electrode and a return electrode of the leadless pacemaker to be delivered by the delivery system. Then, the first mapping electrode and the second mapping electrode are spaced apart from each other like the tip electrode and the return electrode of the implantable leadless pacemaker to be implanted by the delivery system. This enables a particularly high correlation between the value of a mapping signal between the first mapping electrode and the second mapping electrode on the one hand and the value of a signal between the tip electrode and the return electrode of the leadless pacemaker in its implanted state. A particularly appropriate distance lies in a range of from 0.5 cm to 4.5 cm, in particular of from 1.0 cm to 3.5 cm, in particular of from 1.5 cm to 3.0 cm, in particular of from 2.0 cm to 2.5 cm.

In an embodiment, the first mapping electrode and the second mapping electrode are sized and separated in a manner to mimic the size and separation between the tip electrode and the return electrodes of the implantable leadless pacemaker to be delivered by the delivery system. Such an arrangement will particularly enhance the relevance and meaning of impedance and other mapping signals measured between the first mapping electrode and the second mapping electrode with respect to the physiologic conditions between the tip electrode and the return electrode of the implantable leadless pacemaker in its implanted state.

In an embodiment, a first electrode lead for electrically contacting the first mapping electrode and/or a second electrode lead for electrically contacting the second mapping electrode are guided in a sidewall of the catheter device.

In an embodiment, the first mapping electrode is in electric connection with a first contact electrode. In this context, the first contact electrode serves for electrically connecting the tip electrode of a leadless pacemaker device received within the distal end region of the catheter device.

In an embodiment, the first contact electrode is arranged on a contact extension (being another structural element than the mapping extension) that-like the mapping extension also protrudes from the sidewall of the distal end region of the catheter device towards (and at least up to) the central axis of the catheter device. Then, the contact extension can contact a tip electrode of the leadless pacemaker to be implanted in a particularly appropriate manner since the tip electrode is typically also arranged in a region of the central axis of the catheter device.

In an embodiment, the contact extension comprises a shape memory material, such as Nitinol. Then, it is particularly easy to manufacture the contact extension with a pretensioning in a desired direction.

In an embodiment, the contact extension comprises i) an insulating element that surrounds all but the very tip of the contact extension and ii) a conductive electrical connection that runs from the exposed tip back to the catheter device or the mapping extension, respectively. Thus, the contact extension may be designed akin to a wire that has insulation on most of its length excepting an exposed distal tip.

In an embodiment, the mapping extension and the contact extension from a set of cantilevered finger-like extensions. By such an arrangement, both the manufacturing and the use of the catheter device is made possible in a simple way. One of the finger-like extensions (namely the contact extension) electrically contacts the tip electrode of the leadless pacemaker to be implanted by the catheter device. The other of the finger-like extensions (namely the mapping extension) contacts the intended implantation site on the tissue of the patient. Then, the mapping extension and the contact extension form a direct electrical contact between the tip electrode and the tissue at the intended site of implantation without injuring the tissue.

In an embodiment, the second mapping electrode is in electrical connection with the second contact electrode. In this context, the second contact electrode is configured to electrically connect a return electrode (counter electrode) of a leadless pacemaker device received within the lumen in the distal end region of the catheter device.

It is particularly appropriate if the first mapping electrode is in electric contact with a tip electrode of the leadless pacemaker to be implanted and if the second mapping electrode is in electric contact with a return electrode of the same leadless pacemaker to be implanted. Then, the mapping can be performed by addressing only the leadless pacemaker to be implanted without any necessity of an electric circuitry in the catheter device (except for the first mapping electrode, the second mapping electrode and electric connections to the respective electrodes of the leadless pacemaker to be implanted).

Expressed in other words, the leadless pacemaker to be implanted (i.e., the implant) itself can be used to execute the follow-up testing by means of the standard instructions and interfacing from a programmer device GUI (eliminating any need for such features to be replicated in the catheter or pipelined into the programmer device via catheter-enabled conduits). The electrode connections enabled by the catheter effectively extend the implant electrodes to the surface of the catheter such that follow-up routines can be run without having to extend the implant anchor.

In an embodiment, the second contact electrode comprises one or more electrically conductive bulges or bumps protruding from the sidewall of the distal end region of the catheter device towards the lumen of the catheter device. These bulges/bumps serve to establish an electrical connection to a ring electrode of the leadless pacemaker device to be implanted. This ring electrode serves as return electrode for the tip electrode of the leadless pacemaker device and is located more proximally than the stimulation or tip electrode of the leadless pacemaker device. In an embodiment, the electric connection between the bulges/bumps and the ring electrode is realized via a wired connection bridge. The ring electrode brings the implant's return electrode to a position in direct contact with the patient's blood volume. It is worth noting that this direct contact with the patient's blood volume is established at a position/location along the catheter that preserves the tip/return electrode separation found on the implant itself. As such, these bump/bulges are, in an embodiment, not direct electrical feedthroughs but instead feedthroughs that “jog” the location of the implant's return electrode to a position that appropriately mimics the return electrode position for an implanted/deployed leadless pacemaker device.

In an embodiment, the bulge is circumferentially arranged around an inner circumference of the lumen in the distal end region of the catheter device. Then, the bulge defines the position of the leadless pacemaker device to be implanted within the delivery system since the return electrode of the leadless pacemaker device needs to match the bulge in order to establish an electric contact.

In an embodiment, the second mapping electrode and the second contact electrode are spaced apart from each other along the longitudinal extension direction. Such spacing compensates for an offset between the tip electrode of the leadless pacemaker arranged inside the lumen of the distal end region of the catheter device and a distal terminus of the catheter device at which the first mapping electrode is located in the mapping position of the mapping extension. Due to the spacing between the second mapping electrode and the second contact electrode, it is possible that the first mapping electrode and the second mapping electrode are spaced apart from each other in the same distance like the tip electrode and the return electrode of a leadless pacemaker received within the lumen of the catheter device.

In an independently claimed aspect, the present invention relates to a delivery system that can be described in the following way:
A delivery system for placement of a leadless pacemaker device in a human body, the delivery system comprising:
a delivery catheter 4 insertion into a human body, said catheter device having a lumen and a distal end region to be inserted into the human body, the lumen being configured to receive a leadless pacemaker device in the distal end region of the catheter device; and
a first mapping electrode and a second mapping electrode disposed in the distal end region of the delivery catheter 4 sensing, in a mapping mode, a mapping signal between the first mapping electrode and the second mapping electrode; and
wherein the first mapping electrode and the second mapping electrode are arranged on an outside of a sidewall in the distal end region of the catheter device, wherein the first mapping electrode is more distally positioned than the second mapping electrode.

In an embodiment, the first mapping electrode is positioned on a far distal end (directly at the distal terminus) of the distal end region of the catheter device (i.e. on a far distal end of a protector cup serving for housing the leadless pacemaker device to be implanted by the delivery system).

In an embodiment, the first mapping electrode is located in a single area of the outside of the sidewall of the catheter device occupying less than 20°, in particular less than 15°, in particular less than 10°, in particular less than 5° of the circumference of the sidewall, wherein the second mapping electrode is formed as a ring electrode around the whole circumference of the sidewall of the catheter device.

In an embodiment, the first mapping electrode is located in a single area of the outside of the sidewall of the catheter device occupying an area lying in a range of from 0.5 mm² to 5 mm², in particular of from 1 mm² to 4.5 mm², in particular of from 1.5 mm² to 4 mm², in particular of from 2 mm² to 3.5 mm², in particular of from 2.5 mm² to 3 mm², wherein the second mapping electrode is formed as a ring electrode around the whole circumference of the sidewall of the catheter device.

In an aspect, the present invention relates to a pacemaker arrangement comprising one of the delivery systems according to the preceding explanations as well as a leadless pacemaker device received within the lumen in the distal end region of the catheter device. This leadless pacemaker device is intended to be implanted using the delivery system.

In an embodiment, a tip electrode of the leadless pacemaker device is in electric connection with the first mapping electrode by a first contact electrode.

In an embodiment, a return electrode of the leadless pacemaker device is in electric contact with the second mapping electrode via a second contact electrode. In an embodiment, the return electrode is designed as a ring electrode, in particular in a proximal region of the leadless pacemaker device.

In an aspect, the present invention relates to a method of mapping a tissue region for identifying an appropriate implantation site for a leadless pacemaker device. This method comprises the steps explained in the following.

First, a catheter device of the delivery system is at least partially introduced into a human or animal body. In an embodiment, the delivery system is one of the delivery systems as explained above. The catheter device has a lumen and a distal end region. The leadless pacemaker device is received within the lumen in the distal end region of the catheter device. The catheter device further comprises a mapping extension that protrudes from a sidewall of the distal end region of the catheter device towards a central axis of the catheter device. The central axis extends in a longitudinal extension direction of the catheter device. The mapping extension is movable with respect to the sidewall of the distal end region of the catheter device. A first mapping electrode is arranged on the mapping extension. The catheter device further comprises a second mapping electrode that is arranged on an outside of a sidewall of the catheter device in the distal end region of the catheter device.

In another method step, the distal end region of the catheter device is advanced to a tissue site intended for implantation of the leadless pacemaker device.

In a further method step, a terminus of the distal end region of the catheter device is pushed against the intended tissue site of implantation. This results in transferring the mapping extension from a non-mapping position into a mapping position. An angle between the mapping extension and a virtual plane extending perpendicular to the central axis is bigger than 5° in the non-mapping position. The virtual plane intersects the sidewall of the delivery catheter at that site at which the mapping extension is connected to the sidewall. In an embodiment, the angle lies in a range of from 5° to 90°, in particular of from 10° to 85°, in particular of from 15° to 80°, in particular of from 20° to 70°, in particular of from 25° to 60°, in particular of from 30° to 50°, in particular of from 35° to 40°.

In the mapping position, the angle between the mapping extension and the virtual plane lies in a range of from 0° to 5°, in particular of from 1° to 4°, in particular of from 2° to 3°. I.e., in the mapping position, the mapping extension is arranged essentially perpendicular to the sidewall of the distal end region of the catheter device, provided that the sidewall extends essentially parallel to the longitudinal axis (and thus perpendicular to the virtual plane).

In a further method step, a mapping signal is collected by measurements taken across the first mapping electrode and the second mapping electrode. The collected signaling (which includes, in an embodiment, one or more of a sensing input, an impedance input, and/or a pacing capture threshold) is indicative for a suitability of the intended tissue site for an implantation of the leadless pacemaker device.

As noted above, a particularly appropriate mapping signal is a sensing test signal (i.e., an electric signal indicative for intrinsic cardiac activity), an impedance test signal, and/or a pacing capture threshold test signal (i.e., an electric signal indicative for a cardiac response to a pacing pulse).

As already explained above with respect to the delivery system, the described method of mapping the tissue region that makes use of such a delivery system is a particular physiologic and atraumatic possibility of identifying an appropriate implantation site for a leadless pacemaker. The physiologic response of the tissue at the intended implantation site to a stimulus indicates the electric susceptibility of the tissue at the site for later stimulation by a pacing signal of a leadless pacemaker to be implanted. The intended implantation site will be typically found within the heart of the human or animal patient, e.g., in the myocardium of the right ventricle or in the region of the His bundle.

In an independently claimed aspect, the present invention relates to another method of mapping a tissue region for identifying an appropriate implantation site for a leadless pacemaker device. This method can be described in the following way:
A method of mapping a tissue region for identifying an appropriate implantation site for a leadless pacemaker device, the method comprising the following steps:
   a) introducing a catheter device of a delivery system, in particular of the delivery system according to the above explanations, at least partially into a human or animal body, said catheter device having a lumen and a distal end region, wherein a leadless pacemaker device is received within the lumen in the distal end region of the catheter device, wherein a first mapping electrode and a second mapping electrode are disposed in the distal end region of the delivery catheter 4 sensing, in a mapping mode, a mapping signal between the first mapping electrode and the second mapping electrode, wherein the first mapping electrode and the second mapping electrode are arranged on an outside of a sidewall of the catheter device in the distal end region of the catheter device, wherein the first mapping electrode is more distally positioned than the second mapping electrode;
   b) advancing the distal end region of the catheter device to a tissue site intended for implantation of the leadless pacemaker device;
   c) pushing a terminus of the distal end region of the catheter device against the intended tissue site of implantation; and
   d) sensing a mapping signal between the first mapping electrode and the second mapping electrode, the sensed signal being indicative for a suitability of the intended tissue site for an implantation of the leadless pacemaker device.

All embodiments of the delivery systems can be combined in any desired way and can be transferred either individually or in any arbitrary combination to the respective other delivery system, to the pacemaker arrangement, and to the methods of mapping a tissue region. Likewise, all embodiments of the pacemaker arrangement can be combined in in any desired manner and can be transferred either individually or in any arbitrary combination to the described delivery systems and to the methods of mapping a tissue region. Finally, all embodiments of the methods of mapping a tissue region can be combined in any desired way and can be transferred either individually or in any arbitrary combination to the respective other method, to the described delivery systems, and to the pacemaker arrangement.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further details of aspects of the present invention will be explained in the following making reference to exemplary embodiments and accompanying Figures. In the Figures:

FIG. 1A shows a first embodiment of a pacemaker arrangement in a first operational state;

FIG. 1B shows the pacemaker arrangement of FIG. 1A in a second operational state;

FIG. 1C shows the pacemaker arrangement of FIG. 1A in a third operational state;

FIG. 1D shows an enlarged view of FIG. 1B to illustrate the wiring of the pacemaker arrangement;

FIG. 2A shows a second embodiment of a pacemaker arrangement in a first operational state;

FIG. 2B shows the pacemaker arrangement of FIG. 2A in a second operational state;

FIG. 2C shows the pacemaker arrangement of FIG. 2A in the third operational state;

FIG. 2D shows an enlarged view of FIG. 2B to illustrate the wiring of the pacemaker arrangement;

FIG. 2E shows a third embodiment of a pacemaker arrangement;

FIGS. 3A to 3F show a sequence of steps to be performed for loading the catheter device of the pacemaker arrangement of FIG. 2A with an implantable cardiac pacemaker; and

FIG. 4 shows a fourth embodiment of a pacemaker arrangement.

### DETAILED DESCRIPTION

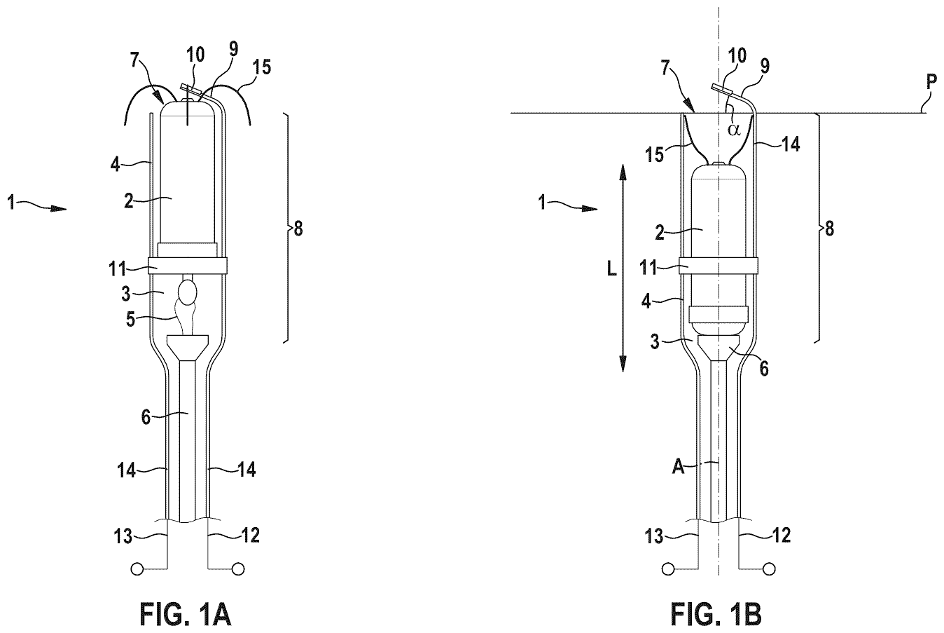

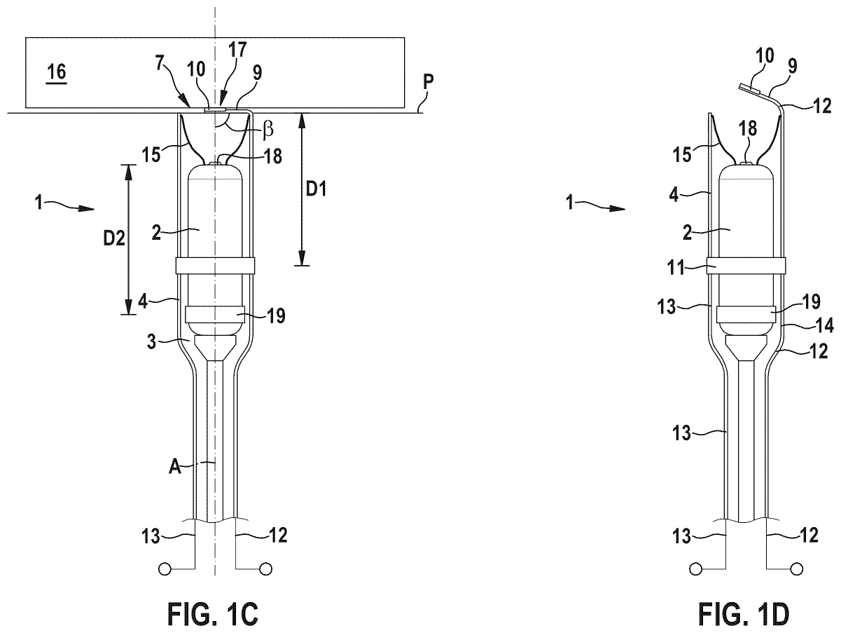

FIG. 1A shows a partially sectional view through a pacemaker arrangement 1 comprising a pacemaker 2 located within a lumen 3 of a delivery catheter 4 serving as catheter device. A traction wire 5 is guided through a guiding element 6 of the delivery system to pull the leadless pacemaker 2 through an opening at a distal terminus 7 of the delivery catheter 4 into the lumen 3 in a distal end region 8 of the delivery catheter 4.

In the area of the distal terminus 7 of the delivery catheter 4, the delivery catheter 4 comprises a mapping extension 9 with a first mapping electrode 10 located at a terminal region of the mapping extension 9. The delivery catheter 4 further comprises a second mapping electrode 11 that is also located in the distal end region 8 of the delivery catheter 4, but proximally of the distal terminus 7. The first mapping electrode 10 is electrically connected with a first mapping electrode lead 12. Likewise, the second mapping electrode 11 is electrically connected with a second mapping electrode lead 13. The first mapping electrode lead 12 and the second mapping electrode 13 are guided through a sidewall 14 of the delivery catheter 4. The second mapping electrode 11 is arranged on an outer surface of the sidewall 14.

FIG. 1B shows the pacemaker arrangement 1 of FIG. 1A in another operational state. In this and in all following Figures, similar elements will be referred to with the same numeral references.

In this second operational state, the leadless pacemaker 2 is received within the lumen 3 of the delivery catheter 4 in its delivery state such that its anchors 15 (or fixation mechanism) are not exposed (i.e., they are not extended from the distal terminus 7 of the delivery catheter 4). In this delivery state, the catheter arrangement 1 can be guided through a human or animal patient to the intended site of implantation of the leadless pacemaker 2 without risking traumatic interactions with patient physiology stemming from exposed, sharp anchors 15. In this operational state, the mapping extension 9 protrudes from the sidewall 14 of the delivery catheter 4 at an angle α of approximately 20° between the mapping 9 extension and a virtual plane P extending perpendicular to a central axis A that extends in a longitudinal extension direction L of the delivery catheter 4 of approximately 20°. The virtual plane P intersects the sidewall 14 of the delivery catheter 4 at that site at which the mapping extension 9 is connected to the sidewall 14. The first mapping electrode 10 intersects the central axis A.

Summarizing, the leadless pacemaker 2 comprises mechanical anchors 15 that lie outside of the delivery catheter 4 in the operational state shown in FIG. 1A during the loading of the delivery catheter 4 with the leadless pacemaker 2, but which are securely positioned within the lumen 3 in the distal end region 8 of the delivery catheter 4 in the operational state shown in FIG. 1B.

FIG. 1C shows another operational state of the pacemaker arrangement, namely a mapping state. In this mapping state, the delivery catheter 4 is pushed against a tissue 16 at an intended implantation site 17. In doing so, the mapping extension 9 is parallel to the virtual plane P so that the angle α is approximately 0°. At the same time, the mapping extension forms an angle β of approximately 90° with the central axis A. As a result, the first mapping electrode 10 tightly abuts the implantation site 17, i.e., without injuring the tissue 16 at the implantation site 17. The implantation site 17 as accessed by the mapping extension 9 is nominally the same location as where the implant's tip electrode 18 will ultimately engage with patient physiology after anchoring the device within the myocardium. The mechanical anchors 15 of the leadless pacemaker 2 still remain within the lumen 3 of the delivery catheter 4 and thus do also not injure the tissue 16 around the implantation site 17.

Also, in this mapping state, the first mapping electrode 10 intersects the central axis A. Furthermore, a tip electrode 18 of the implantable pacemaker 2 intersects the central axis A. Thus, if the implantable pacemaker 2 is implanted, the tip electrode 18 will be located at the same position in which the first mapping electrode 10 non-invasively maps the susceptibility of the tissue 16 at the implantation site 17 for getting electrically stimulated by a pacing pulse. A first distance D1 between the first mapping electrode 10 (when the mapping extension 9 is in its mapping position) and the second mapping electrode 11 equals a second distance D2 between the tip electrode 18 of the leadless pacemaker 2 and a return electrode 19 of the leadless pacemaker 2. This return electrode 19 is designed as a ring electrode at a proximal end of the leadless pacemaker 2.

Matching this distance or separation, in particular along with the effective electrode sizes of the implantable pacemaker 2 means that the delivery catheter 4 can approximate the electrical engagements of the implantable pacemaker 2 with the patient's conduction system and/or stimulatable tissue without having to deploy the mechanical anchors 15 of the implantable pacemaker 2. This aids in finding a viable electrical interface with the patient's conduction system without having to first physically pierce the patient's anatomy.

In the mapping state shown in FIG. 1C, the mapping extension 9 is essentially flush with the distal terminus 7 of the delivery catheter 4. It does essentially not protrude from the distal terminus 7 along the central axis A.

FIG. 1D shows an enlarged view of FIG. 1B to illustrate the wiring of the pacemaker arrangement 1. It can well be seen that the first mapping electrode lead 12 extends inside the whole length of the sidewall 14 of the delivery catheter 4 as well as inside the mapping extension 9 to finally electrically contact the first mapping electrode 10. Thus, the first electrode lead 12 electrically connects the first mapping electrode 10 with a corresponding first mapping electrode port.

Likewise, the second mapping electrode lead 13 is guided inside the sidewall 14 of the delivery catheter 4 up until the second mapping electrode 11. The second electrode lead 13 electrically connects the second mapping electrode 11 with a corresponding second mapping electrode port.

The first mapping electrode lead 12 and the second mapping electrode lead 13 are electrically insulated against each other. It is immediately apparent that the first electrode lead 12 does not contact the second mapping electrode 11, and that the second electrode lead 13 does not contact the first mapping electrode 10.

In this embodiment, neither the tip electrode 18 nor the return electrode 19 of the implantable pacemaker 2 (cf. FIG. 1C for more details on these elements) are in electrical contact with the first mapping electrode 10 or the second mapping electrode 11. Rather, the pacemaker arrangement 1 shown in FIGS. 1B and 1D is an embodiment in which the mapping is conducted by the catheter device 4, not by the implantable pacemaker 2.

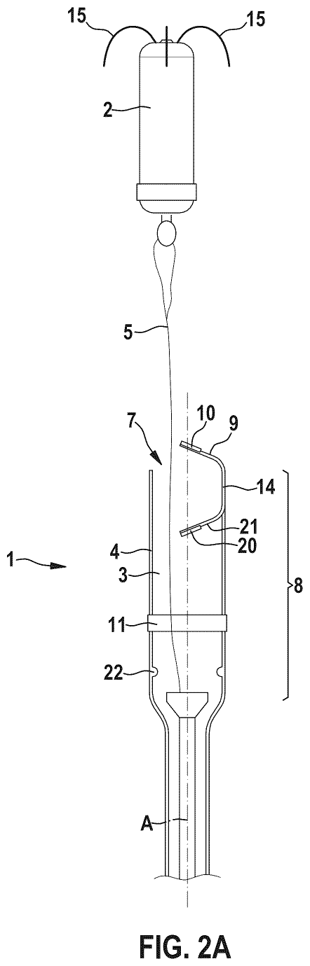

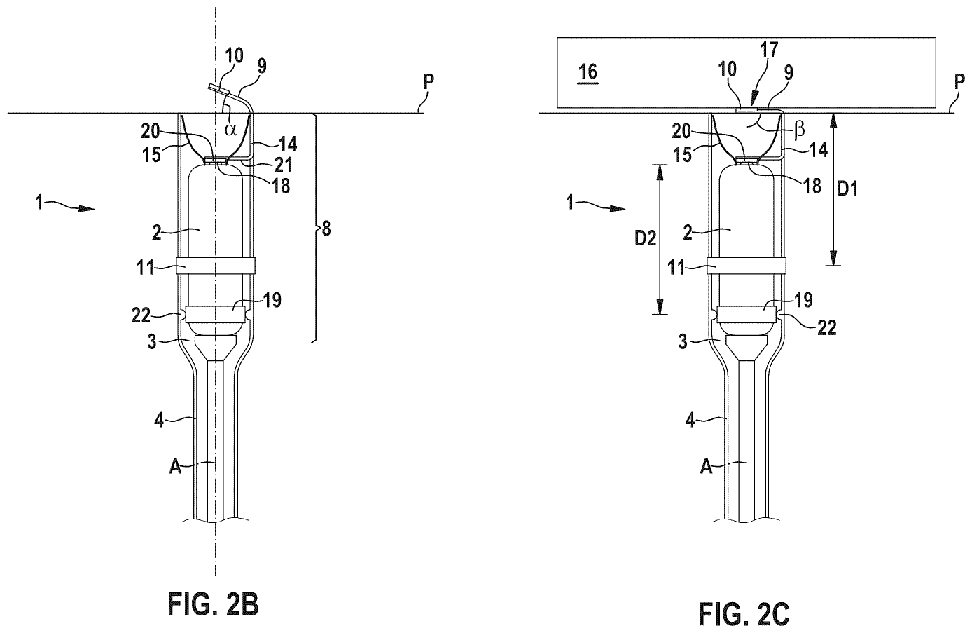

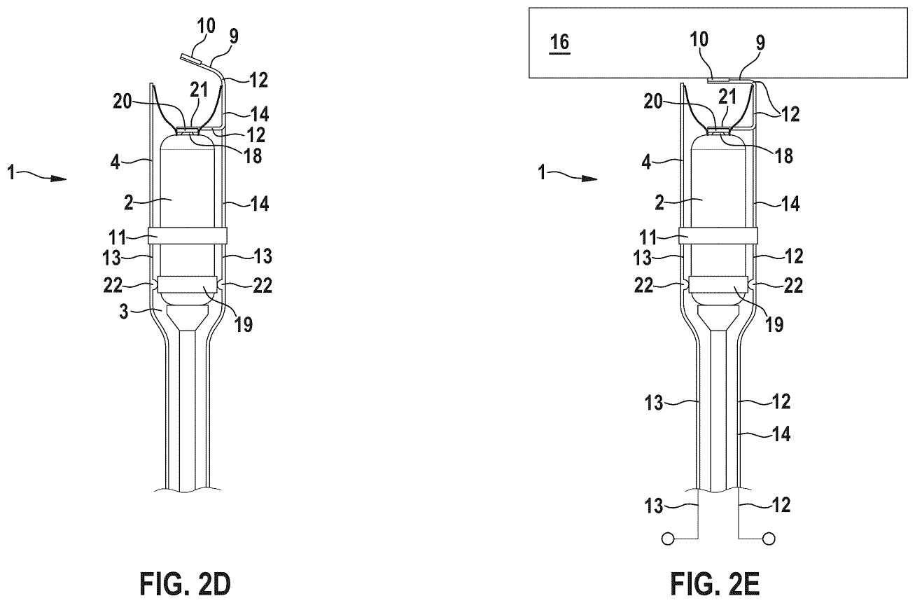

FIG. 2A shows a second embodiment of a pacemaker arrangement 1 in a first operational state in which the leadless pacemaker 2 is pulled by a traction wire 5 into a lumen 3 of a delivery catheter 4. The leadless pacemaker 2 comprises mechanical anchors 15 that are in their deployed state. The delivery catheter 4 comprises a mapping extension 9 that extends at the distal terminus 7 from a sidewall 14 of the delivery catheter 4 towards a central axis A of the delivery catheter 4. The mapping extension 9 comprises a first mapping electrode 10 that is an electric contact with a contact electrode 20. The contact electrode 20 is located on a contact extension 21 that also extends from the sidewall 14 towards the central axis A. The mapping extension 9 and the contact extension 21 form a set of cantilevered fingerlike extensions. At a proximal side of the distal end region 8 of the delivery catheter 4, a bulge 22 is arranged that protrudes from the sidewall 14 into the lumen 3 of the delivery catheter 4. The bulge 22 is electrically conductive and is in electric contact with the second mapping electrode 11.

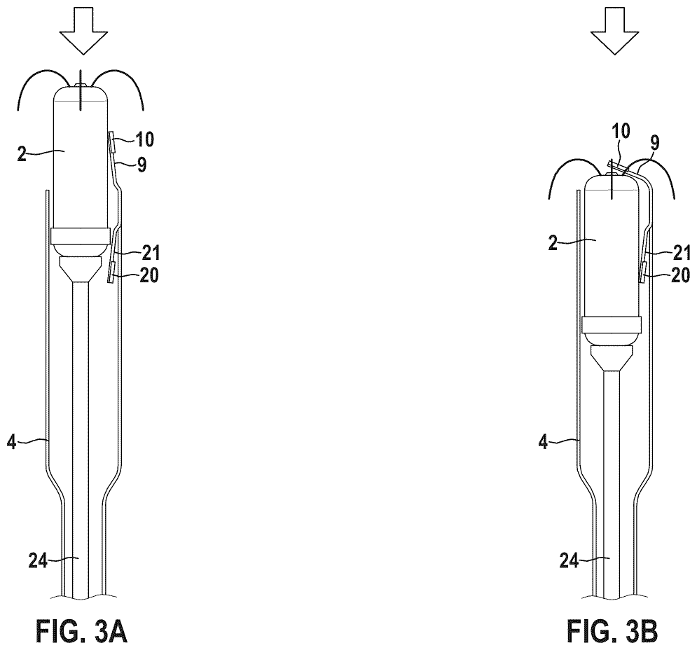

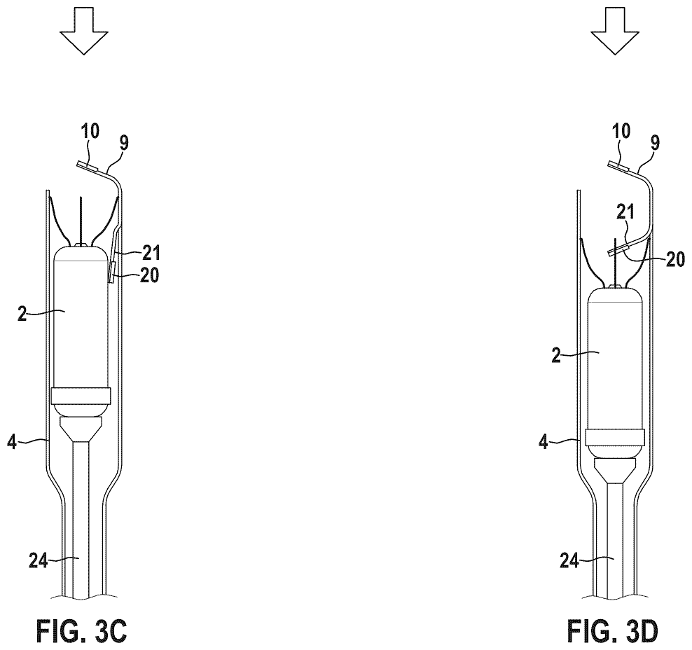

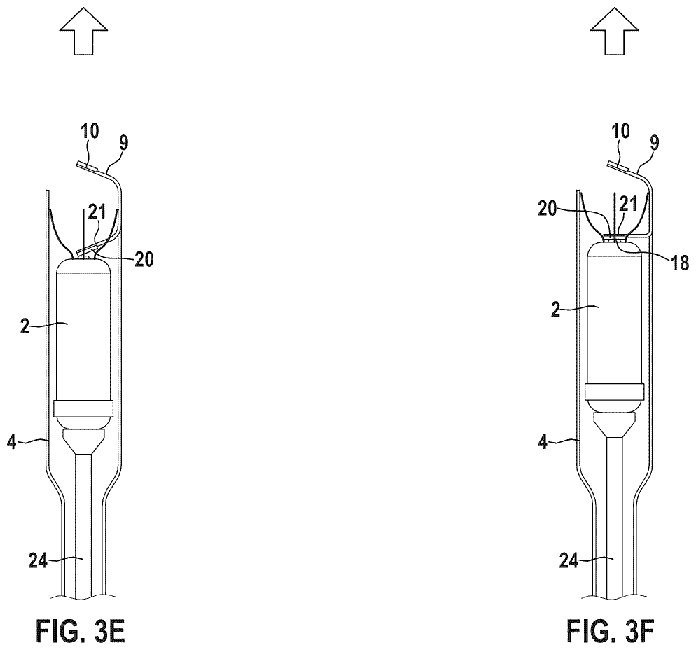

Once the leadless pacemaker 2 has reached its intended position within the lumen 3 in the distal end region 8 of the delivery catheter 4, as shown in FIG. 2B (which would nominally demand that the implant be withdrawn internal to the distal tip of the catheter to an extent that it first could permit passing the full length of the implant body proximal to the contact extension 21 such that the contact extension could return to a natural flexure position as in FIG. 2A after which the implant would be advanced in a distal direction within the catheter to realize the intended configuration in FIG. 2B; cf. FIGS. 3A to 3F for more details), the first contact electrode 20 establishes an electric contact with a tip electrode 18 of the leadless pacemaker 2. Due to a pretensioning of the contact extension 21 in the proximal direction of the delivery catheter 4, the contact element 21 firmly pushes with its contact electrode 20 onto the tip electrode 18 of the leadless pacemaker 2. Consequently, there is a direct electric contact between the tip electrode 18 and the first mapping electrode 10.

The mapping extension 9 protrudes from the sidewall 14 of the delivery catheter 4 at an angle α of about 20° between the mapping extension 9 and the virtual plane P towards the central axis A. The bulge 22 electrically contacts a return electrode 19 of the leadless pacemaker 2. Consequently, there is also a direct electric contact between the return electrode 19 and the second mapping electrode 11.

FIG. 2C shows the pacemaker arrangement 1 of FIGS. 2A and 2B in a mapping position, i.e., when being pushed against tissue 16 at an intended implantation site 17. Reference is made in this respect to the explanations given with respect to FIG. 1C excepting the fact that in FIG. 1C the mapping can only be conducted by the delivery catheter 4 itself while in FIG. 2C the mapping can be administered by the leadless pacemaker 2 in combination with the delivery catheter 4.

Like in case of the embodiment shown in FIG. 1C, a distance D1 between the first mapping electrode 10 and the second mapping electrode 11 corresponds to (is equal to) a second distance D2 between the tip electrode 18 and the return electrode 19 of the leadless pacemaker 2. Since the first mapping electrode 10 and the second mapping electrode 11 are in direct electric contact with the tip electrode 18 or the return electrode 19, respectively, all electric tests with respect to the suitability of the implantation site 17 for a later implantation of the leadless pacemaker 2 can be directly performed with the leadless pacemaker 2 (distinguishing it from the capabilities offered by the embodiment in FIG. 1). Thus, an external programmer addressing the delivery catheter 4 is no longer necessary in this embodiment. Rather, the leadless pacemaker 2 can be directly addressed and can directly provide and receive the electric signals necessary for the tests to be performed.

The mapping extension 9 abuts the tissue 16 approximately parallel. Thus, the mapping extension 9 is parallel with the virtual plane P so the angle α is approximately 0°. Consequently, the mapping extension forms an angle β of approximately 90° with the central axis A.

FIG. 2D shows an enlarged view of FIG. 2B to illustrate the wiring of the pacemaker arrangement 1.

It can well be seen that the first mapping electrode lead 12 extends only from the contact electrode 20 inside the contact extension 21, inside a small portion of the sidewall 14 of the delivery catheter 4 as well as inside the mapping extension 9 to the first mapping electrode 10. In contrast to the embodiment shown in FIGS. 1B and 1D, the first mapping electrode lead does not extend along the whole length of the sidewall 14 of the delivery catheter 4. Thus, in this embodiment, the first electrode lead 12 electrically connects the first mapping electrode 10 with the tip electrode 18 of the leadless pacemaker 2.

The second mapping electrode lead 13 is guided inside the sidewall 14 of the delivery catheter 4 between the bulge 22, which electrically contacts the return electrode 19 of the leadless pacemaker 2, up until the second mapping electrode 11. The second electrode lead 13 electrically connects the second mapping electrode 11 with the return electrode 19 of the leadless pacemaker 2.

The first mapping electrode lead 12 and the second mapping electrode lead 13 are electrically insulated against each other. It is immediately apparent that the first electrode lead 12 does not contact the second mapping electrode 11, and that the second electrode lead 13 does not contact the first mapping electrode 10.

In this embodiment, the tip electrode 18 and the return electrode 19 of the leadless pacemaker 2 are in electrical contact with the first mapping electrode 10 or the second mapping electrode 11, respectively. Consequently, the pacemaker arrangement 1 shown in FIGS. 2B and 2D is an embodiment in which the mapping is nominally conducted by the implantable pacemaker 2, not by the catheter device 4 which only serves for extending the tip electrode 18 and the return electrode 19 of the leadless pacemaker 2 to their intended sites of action.

FIG. 2E shows a third embodiment of the leadless pacemaker arrangement 1 representing a combination of the embodiments shown in FIGS. 1A to 1D on the one hand and in FIGS. 2A to 2D on the other hand.

It can well be seen that the first mapping electrode lead 12 extends inside the whole length of the sidewall 14 of the delivery catheter 4 as well as inside the mapping extension 9 to finally electrically contact the first mapping electrode 10. The first mapping electrode lead 12 also extends from the contact electrode 20 inside the contact extension 21, inside a small portion of the sidewall 14 of the delivery catheter 4 as well as inside the mapping extension 9 to the first mapping electrode 10. Thus, in this embodiment, the first electrode lead 12 electrically connects the first mapping electrode 10 with a corresponding first mapping electrode port and with the tip electrode 18 of the leadless pacemaker 2. Appropriate electrode separations for choosing either electrical path are not shown for simplification.

The second mapping electrode lead 13 is guided inside the sidewall 14 of the delivery catheter 4 from a second mapping electrode port up until the second mapping electrode 11. It is furthermore guided inside the sidewall 14 of the delivery catheter 4 between the bulge 22, which electrically contacts the return electrode 19 of the leadless pacemaker 2, up until the second mapping electrode 11. The second electrode lead 13 electrically connects the second mapping electrode 11 with the second mapping electrode port and with the return electrode 19 of the leadless pacemaker 2. Once again, appropriate electrode separations for choosing either electrical path are not shown for simplification.

In this embodiment, it can be decided by the user whether mapping is to be conducted i) by the leadless pacemaker 2 (via its tip electrode 18 and is return electrode 19 that are in electrical connection with the first mapping electrode 10 or the second mapping electrode 11, respectively) or ii) by the delivery catheter 4, i.e., via the first mapping electrode port and the second mapping electrode port connected to the delivery catheter 4.

Thus, the pacemaker arrangement 1 shown in FIG. 2E is an embodiment in which the mapping can be conducted by the implantable pacemaker 2 or by the catheter device 4.

For sake of completeness, also in this embodiment, the first mapping electrode lead 12 and the second mapping electrode lead 13 are electrically insulated against each other. It is immediately apparent that the first electrode lead 12 does not contact the second mapping electrode 11, and that the second electrode lead 13 does not contact the first mapping electrode 10.

FIGS. 3A to 3F show a sequence of method steps to be performed for loading the leadless pacemaker 2 of the embodiment shown in FIGS. 2A to 2D into the delivery catheter 4. The arrows shown in top of each of FIGS. 3A to 3F indicate the direction of movement to be performed in the individual method steps. A downward arrow indicates a movement in the proximal direction of the delivery catheter 4, and an upward arrow indicates a movement in the distal direction of the delivery catheter 4.

In FIG. 3A, the leadless pacemaker 2 is pulled with a loading device 24 into the lumen of the catheter device 4. The mapping extension 9 is bent distally and the contact extension 21 is bent proximally. As can be seen in FIG. 3B, the leadless pacemaker 2 is further pulled in the proximal direction of the delivery catheter 4. In the state illustrated in FIG. 3B, the leadless pacemaker 2 still contacts the mapping extension 9. When the leadless pacemaker 2 is further pulled in the proximal direction of the delivery catheter 4, it loses contact with the mapping extension 9 (cf. FIG. 3C). As illustrated in FIG. 3D, the leadless pacemaker 2 is pulled that far into the lumen of the delivery catheter 4 until it also loses contact with the contact extension 21 (cf. FIG. 3D). Therefore, it is necessary that the distal region of the delivery catheter 4 is longer than the leadless pacemaker 2 (in each case considered along the longitudinal extension direction of the delivery catheter 4).

Afterwards, the leadless pacemaker 2 is pushed with the loading device 24 in the distal direction, as shown in FIG. 3E. This movement in the distal direction is continued until the leadless pacemaker 2 comes into close contact with the contact extension 21, as shown in FIG. 3F. Now, the contact electrode 20 located on the contact extension 21 firmly abuts the tip electrode 18 of the leadless pacemaker 2 and establishes an electrical contact between the first mapping electrode 10 located on the mapping extension 9 and the tip electrode 18 of the leadless pacemaker 2. The state illustrated in FIG. 3F corresponds to the state of the pacemaker arrangement 1 shown in FIG. 2B.

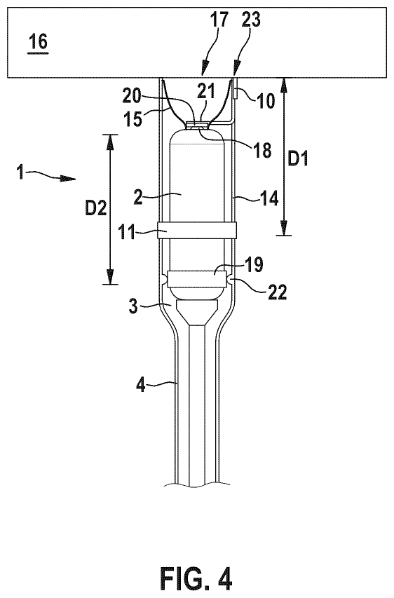

FIG. 4 shows another embodiment of a pacemaker arrangement 1. This pacemaker arrangement 1 is similar to the pacemaker arrangement shown in FIGS. 2A to 2C. Therefore, reference is also made to the explanations given above with respect to FIGS. 2A to 2C The pacemaker arrangement 1 of FIG. 4 also comprises a contact extension 21 for directly contacting a tip electrode 18 of a leadless pacemaker 2 received within a lumen 3 of a delivery catheter 4. However, in contrast to the embodiment shown in FIGS. 2A to 2C, a first mapping electrode 10 is not arranged on a mapping extension, but rather directly on an outside of a sidewall 14 of the delivery catheter 4.

Such a condition makes the delivery catheter 4 easier to load compared to FIG. 2 embodiments, but introduces an offset between a tissue test site 23 (i.e., the site where mapping data can be gathered) versus where the tip electrode 18 of the leadless pacemaker 2 will ultimately interface with the patient's tissue 17 upon implantation. Therefore, the results of the test of the suitability of the tissue 16 for an implantation of the leadless pacemaker 2 as supported by embodiments found in FIG. 4 will not exactly reflect the suitability of the tissue at the implantation site 17, but will rather be an approximation thereof.

While a distance D1 between the first mapping electrode 10 and a second mapping electrode 11 still matches a second distance D2 between a tip electrode 18 and a return electrode 19 of the leadless pacemaker 2 to be implanted, the measurements done with the embodiment shown in FIG. 4 will be somewhat less accurate than the measurements done with the embodiments shown in FIGS. 1A to 2C. However, as noted above, by placing the first mapping electrode 10 to an outside of the sidewall 14 of the delivery catheter 4 its construction becomes less demanding than when placing the first mapping electrode 10 on a mapping extension like in case of the embodiments shown in FIGS. 1A to 2C. With the contact extension 21 being the only internal finger-like extension of the delivery system 1, the delivery catheter 4 may likely be repositioned (a process that demands the deployment and resheathing of, at a minimum the anchors 15 of the leadless pacemaker 2—and more likely the entire body length of the leadless pacemaker 2—by the distal end of the catheter) more readily than the delivery catheter of the embodiment shown in FIGS. 2A to 2C.

The first mapping electrode 10 is directly connected with the tip electrode 18 of the leadless pacemaker 2 due to the contact electrode 20 on the contact extension 21. This contact extension 21 is generally formed like the contact element 21 of the embodiment show in FIGS. 2A to 2C.

Thus, the embodiment of FIG. 4 closely resembles the embodiment shown in FIGS. 2A to 2C and shares most of the properties of that embodiment. The main difference is that the electric susceptibility of the tissue 16 is not directly measured at the intended implantation site 17, but rather at the tissue test site 23 offset from the target location by the nominal radius of the catheter's distal terminus.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A delivery system for placement of a leadless pacemaker device in a human body, the delivery system comprising:
a catheter device for insertion into a human body, said catheter device having a lumen and a distal end region to be inserted into the human body, the lumen being configured to receive a leadless pacemaker device in the distal end region of the catheter device; and
a first mapping electrode and a second mapping electrode disposed in the distal end region of the catheter device for sensing, in a mapping mode, a mapping signal between the first mapping electrode and the second mapping electrode;
wherein the first mapping electrode is arranged on a mapping extension that protrudes from a sidewall of the distal end region of the catheter device towards a central axis of the catheter device, the central axis extending in a longitudinal extension direction of the catheter device;
wherein the mapping extension is movable with respect to the sidewall of the distal end region of the catheter device between a non-mapping position and a mapping position, wherein an angle between the mapping extension and a virtual plane extending perpendicular to the central axis is bigger than 5° in the non-mapping position and lies in a range of from 0° to 5° in the mapping position; and
wherein the second mapping electrode is arranged on an outside of the sidewall in the distal end region of the catheter device.

2. The delivery system according to claim 1, wherein the first mapping electrode and the central axis of the catheter device intersect at least in the mapping position of the mapping extension.

3. The delivery system according to claim 1, wherein the mapping extension protrudes from a distal terminus of the catheter device in the longitudinal extension direction of the catheter device in the non-mapping position and is nominally flush with the distal terminus of the catheter device in the mapping position.

4. The delivery system according to claim 1, wherein a distance between the first mapping electrode and the second mapping electrode in the mapping position of the mapping extension equals a distance between a tip electrode and a return electrode of a leadless pacemaker to be delivered by the delivery system.

5. The delivery system according to claim 1, wherein at least one of a first electrode lead for electrically contacting the first mapping electrode and a second electrode lead for electrically contacting the second mapping electrode are guided in the sidewall of the catheter device.

6. The delivery system according to claim 1, wherein the first mapping electrode is in electric connection with a first contact electrode, the first contact electrode being configured to electrically connect a tip electrode of a leadless pacemaker device received within the lumen in the distal end region of the catheter device.

7. The delivery system according to claim 6, wherein the first contact electrode is arranged on a contact extension that protrudes from the sidewall of the distal end region of the catheter device towards the central axis of the catheter device.

8. The delivery system according to claim 7, wherein the mapping extension and the contact extension form a set of cantilevered finger-like extensions.

9. The delivery system according to claim 1, wherein the second mapping electrode is in electric connection with a second contact electrode, the second contact electrode being configured to electrically connect a return electrode of a leadless pacemaker device received within the lumen in the distal end region of the catheter device.

10. The delivery system according to claim 9, wherein the second contact electrode comprises a bulge protruding from the sidewall of the distal end region of the catheter device towards the lumen of the catheter device.

11. The delivery system according to claim 9, wherein the second mapping electrode and the second contact electrode are spaced apart from each other in the longitudinal extension direction.

12. A pacemaker arrangement comprising the delivery system according to claim 1, and a leadless pacemaker device received within the lumen in the distal end region of the catheter device.

13. The pacemaker arrangement according to claim 12, wherein a tip electrode of the leadless pacemaker device is in electric connection with the first mapping electrode via a first contact electrode.

14. The pacemaker arrangement according to claim 12, wherein a return electrode of the leadless pacemaker device is in electric connection with the second mapping electrode via a second contact electrode.

15. A method of mapping a tissue region for identifying an appropriate implantation site for a leadless pacemaker device, the method comprising the following steps:
a) introducing a catheter device of a delivery system, in particular of the delivery system according to claim 1, at least partially into a human or animal body, said catheter device having a lumen and a distal end region, wherein a leadless pacemaker device is received within the lumen in the distal end region of the catheter device, the catheter device further comprising a mapping extension that protrudes from a sidewall of the distal end region of the catheter device towards a central axis of the catheter device, the central axis extending in a longitudinal extension direction of the catheter device, wherein the mapping extension is movable with respect to the sidewall of the distal end region of the catheter device, wherein a first mapping electrode is arranged on the mapping extension, wherein the catheter device further comprises a second mapping electrode that is arranged on an outside of the sidewall in the distal end region of the catheter device;
b) advancing the distal end region of the catheter device to a tissue site intended for implantation of the leadless pacemaker device;
c) pushing a terminus of the distal end region of the catheter device against the tissue site intended for implantation, thereby transferring the mapping extension from a non-mapping position into a mapping position, wherein an angle between the mapping extension and a virtual plane extending perpendicular to the central axis is bigger than 5° in the non-mapping position and lies in a range of from 0° to 5° in the mapping position; and
d) collecting a mapping signal between the first mapping electrode and the second mapping electrode, the collected signal being indicative for a suitability of the tissue site intended for an implantation of the leadless pacemaker device.
